(19) United States
(12) Patent Application Publication
(10) Pub. No.: US 2024/0359028 A1
(43) Pub. Date: Oct. 31, 2024

(54) RADIATION SOURCE AND SKIN IRRADIATION DEVICE

(71) Applicant: Günter Schaffron, Meppen (DE)

(72) Inventor: Günter Schaffron, Meppen (DE)

(21) Appl. No.: 18/289,275

(22) PCT Filed: Apr. 28, 2022
(86) PCT No.: PCT/EP2022/061348

§ 371 Date: Nov. 2, 2023

(30) Foreign Application Priority Data
May 10, 2021 (EP) 21173008

(51) Int. Cl. A61N 5/06

(52) U.S. Cl. CPC A61N 5/0614; A61N 2005/0615; A61N 2005/0638; A61N 2005/064; A61N 2005/0651; A61N 2005/0661

(57) ABSTRACT

A radiation source for a skin irradiation device for tanning skin, designed to emit UV light at a spectral irradiance E_{λ}(λ), the spectral irradiance E_{λ}(λ) being chosen such that the ratio of the NMSC-effective, biological irradiance E_{NMSC} to the erythema-effective, biological irradiance Eₑᵣ is 0.9 or less.

The invention relates to a radiation source for emitting UV radiation for tanning skin as well as a skin irradiation device comprising such a radiation source.

Low-pressure UV tubes or high-pressure UV burners having filter disks are usually utilized as radiation sources for skin irradiation devices for tanning skin, that is to say for solariums. Thus, low-pressure UV tubes or high-pressure UV burners can, for example, solely be provided as irradiation sources. A combination of low-pressure UV tubes and high-pressure UV burners having filter disks can also be provided, the low-pressure tubes usually being used for irradiating the body region and the high-pressure burners for irradiating the facial region. LED flood lamps can also be utilized for the facial region or even for irradiating the entire body.

Radiation sources for tanning skin usually emit UV radiation in a range between 280 nm and 400 nm wavelength or in subranges thereof. Radiation in the UVA range between 315 nm and 400 nm wavelength has a greater depth of penetration into biological tissue and substantially contributes to direct pigmentation, that is to say, to the development of a short-term tan. By contrast, UVB radiation, in the wavelength range from 280 nm to 315 nm, penetrates less deeply into biological tissue and, as a result, induces a delayed formation of melanin and, therefore, indirect pigmentation, that is to say delayed, longer-term tanning. In order to obtain an optimal tanning result, a finely tuned ratio of UVA to UVB radiation and a defined irradiance are of importance.

The wavelength-dependent actions of UV radiation, in particular on the skin, form the subject of comprehensive investigations. Important definitions can be found, by way of example, in the DIN 5031-10:2018-03 standard. Therein, the radiation striking the skin is characterized by its spectral irradiance E_{λ}(λ). The spectral irradiance E_{λ}(λ) indicates the radiant power striking the skin in a specific wavelength range, as a general rule in watts per square meter. It corresponds to the radiant power arriving from a radiation source.

Another important parameter, with which the biological effects of the radiation striking the skin are described in the standard mentioned, is the so-called photobiologically effective irradiance E_{biol} (hereinafter also referred to as biological irradiance, for short). Knowing the relative spectral sensitivity A_{boil}(λ) of the respective biological process, it can be calculated from the spectral irradiance E_{λ}(λ) as follows:

$${E}_{biol} = \int {E}_{λ} ( λ ) {A}_{biol} ( λ ) d λ$$

The integration is to be carried out over the entire relevant wavelength range. The relative spectral sensitivity A_{boil}(λ) is also referred to as the spectral action factor or as the action spectrum.

Spectral action factors are known for numerous biological effects of UV radiation and are indicated, by way of example, in the standard mentioned, so that the respective biological irradiance can be calculated from a spectral irradiance E_{λ}(λ) of a radiation source, which is known (e.g., measured with a double monochromator). This applies, for example, to the following photobiologically effective irradiances:
erythema-effective irradiance Eₑᵣ. It is responsible for the formation of UV erythema. UV erythema, also referred to below as erythema for short, is the symptom of an acute skin inflammation which is produced by UV irradiation, also referred to as sunburn.
NMSC-effective irradiance E_{NMSC}. It is responsible for the formation of non-melanoma skin cancer. NMSC stands for non-melanoma skin cancer.
immediate pigmentation-effective irradiance Eₚᵢ. It is responsible for the direct pigmentation or immediate pigmentation.

Radiation sources for tanning skin are utilized in private and commercial environments. In both cases, strict conditions apply to the operation which, if they are observed, mean that all of the limit values applicable to health protection are complied with. By way of example, a maximum irradiation time can be stipulated, depending on the skin type, such that no UV erythema is produced. Limit values can also be stipulated and monitored for the irradiation dose obtained, e.g., over the course of a year, by way of example, in order to exclude a significant increase in the risk of developing non-melanoma skin cancer. Consequently, all the desired and undesired effects of the irradiation can be controlled and monitored very well, as a general rule better than when the skin is irradiated by the sun. That is why tanning the skin in a solarium is, nowadays, deemed to be very safe.

The basic outlines of the photobiological effectiveness of UV radiation are described in the printed document “Protection of individuals from the dangers of solar UV radiation and UV radiation in solariums”, recommendation of the German Commission on Radiological Protection in Bonn, by Beate Volkmer et al., adopted at the 280^{th} meeting of the German Commission on Radiological Protection on 11/12 Feb. 2016, XP55852193A.

A UVB radiation source has become known from printed document US 2013/0172963 A1, which is not utilized for tanning skin, but rather to promote vitamin D synthesis. It emits narrow-band UV radiation in a wavelength range around 297 nm, in particular in the range from 292 nm to 302 nm.

Proceeding therefrom, it is the object of the invention to make available a radiation source as well as an irradiation device which, with a sufficient tanning effect, represents a further reduced risk of NMSC cancer.

The object is achieved by the radiation source having the advantages of Claim 1 and by the skin irradiation device having the advantages of Claim 11. Advantageous embodiments are indicated in the subclaims.

The radiation source is intended for a skin irradiation device for tanning skin and is designed to emit UV light at a spectral irradiance E_{λ}(λ), the spectral irradiance E_{λ}(λ) being chosen such that the ratio of the NMSC-effective, biological irradiance E_{NMSC} to the erythema-effective, biological irradiance Eₑᵣ is 0.9 or less.

The radiation source is designed to emit UV light in a wavelength range from 280 nm to 400 nm or in parts thereof. Radiation can be additionally emitted in other wavelength ranges, in particular visible light. The emitted wavelength spectrum corresponds to a spectral irradiance E_{λ}(λ). Said irradiance describes the irradiated power per area and can be indicated in W/m². It depends on the distance between the radiation source and the irradiated area. In practice, said distance is specified by the design constraints of a solarium and corresponds, in particular, to the distance between the radiation source and a skin part to be irradiated. The spectral irradiance E_{λ}(λ) can, in principle, have any distribution, by way of example a largely continuous spectrum which extends over the entire UV range or a combination of individual more or less pronounced peaks. In particular, the spectral irradiance E_{λ}(λ) can have a maximum value, i.e., a global maximum or a local maximum which lies in a wavelength range from 330 nm to 390 nm.

As explained at the outset, the NMSC-effective, biological irradiance E_{NMSC} and the erythema-effective, biological irradiance Eₑᵣ can be calculated from said spectral irradiance E_{λ}(λ). The spectral action factors to be enlisted herefor are generally known in professional circles and can, if required, be inferred from the DIN 5031-10:2018-03 standard cited at the outset, in particular Table A.3 contained therein, which contains details regarding the spectral action factor Aₑᵣ(λ) for the UV erythema, and Table A.6 which contains details regarding the spectral action factor A_{nmsc}(λ) for the NMSC photocarcinogenesis.

A particular feature of the invention is that the wavelength spectrum of the radiation source and, therefore, the spectral irradiance E_{λ}(λ) of the radiation source are chosen such that the ratio between E_{NMSC} and Eₑᵣ is 0.9 or less. Optionally, the ratio can also be kept smaller and can, for example, be 0.8 or less, 0.7 or less, 0.6 or less or 0.5 or less.

Different measures are available to the person skilled in the art for said choice of spectral irradiance E_{λ}(λ). In principle, the spectral irradiance E_{λ}(λ) can be influenced by focusing the production of the UV radiation on specific wavelength ranges. The measures available for this depend on the type of radiation source and are explained in greater detail below. Alternatively, proportions of the generated radiation can be filtered out, e.g., by arranging an appropriate filter between the location of the radiation generation and the skin. To this end, e.g., filter disks which are utilized in connection with high-pressure UV burners, but also special glass or plastic materials which can be installed as part of the housing of the radiation source, are suitable.

The actual connection between the NMSC-effective, biological irradiance and the risk of an individual exposed to said radiation developing non-melanoma skin cancer is not yet exactly known scientifically. The published action factors are based on data from experiments on mice, which have been projected and extrapolated to the UV transmission of human skin. However, it is not disputed that a higher NMSC-effective irradiance is associated with a higher risk.

In connection therewith, the inventor has recognized that the action spectra for the UV erythema and for the NMSC photocarcinogenesis do indeed overlap one another significantly, but differ sufficiently from one another such that, with a suitable choice of spectral irradiance E_{λ}(λ) of the radiation source, it is possible to always keep the NMSC-effective irradiance noticeably lower than the erythema-effective irradiance.

This leads to the fact that an excessively high irradiation dose will make itself felt in the form of sunburn due to the relatively high erythema effectiveness before a high NMSC risk arises. In the case of moderate irradiation which does not lead to UV erythema, the NMSC-effective biological irradiation also remains relatively low. That is to say that the result is that the NMSC risk can be kept particularly low by deliberately choosing the spectral irradiance E_{λ}(λ) of the radiation source. This applies both compared to irradiation with sunlight which typically has a ratio between E_{NMSC} and Eₑᵣ of approximately 2.2, and compared to commercially available irradiation devices, in the case of which the inventor has determined ratios between E_{NMSC} and Eₑᵣ of at least 1.1, but, as a general rule, 1.4 or more, depending on the design.

A further advantage is that, in practice, the irradiation dose is constantly specified on the basis of the erythema-effective irradiance. For this purpose, the radiation sources, in this case low-pressure tubes, are equipped with a so-called UV key which comprises two numerical values designated as X and Y. The X value indicates the erythema-effective irradiance in the case of a free-burning lamp at a distance of 25 cm in mW/m². The maximum irradiation time is stipulated, taking into account the actual distance between the skin and radiation source, and the respective skin type. In the case of said procedure, it is ensured by the invention that the NMSC-effective irradiance is likewise kept low without further action.

In one embodiment, the spectral irradiance E_{λ}(λ) has a maximum value in the wavelength range from 330 nm to 390 nm, and has values in the wavelength range from 299 nm to 311 nm and in the wavelength range from 315 nm to 329 nm, which do not exceed 5% of said maximum value. Optionally, the wavelength range, in which the limit of 5% of the maximum value is not exceeded, can also already begin at 296 nm, that is to say comprise the ranges of 296 nm to 311 nm and of 315 nm to 329 nm. The limit of 5% can, optionally, also be chosen to be lower, for example 3.5% of the maximum value, 2% of the maximum value or even 1% of the maximum value. Said limitations of the spectral irradiance E_{λ}(λ) contribute to keeping the ratio between E_{NMSC} and Eₑᵣ low, because the spectral action factor A_{NMSC}(λ) for the NMSC photocarcinogenesis is greater in the wavelength ranges mentioned than the spectral action factor Aₑᵣ(λ) for the UV erythema. It is true that the wavelength range between 311 nm and 315 nm, which is excluded from the restriction in said embodiment, likewise has an unfavorable ratio between the two spectral action factors, but in the case of some radiation sources, the emission spectrum of which has a mercury peak at 313 nm, it has a higher spectral irradiance E_{λ}(λ), the suppression of which would require additional measures and can be accepted as long as the desired ratio between E_{NMSC} and Eₑᵣ is complied with.

In one embodiment, the spectral irradiance E_{λ}(λ) has a maximum value in the wavelength range from 330 nm to 390 nm, and has values in the wavelength range from 299 nm to 329 nm, which do not exceed 5% of said maximum value. In said embodiment as well, the wavelength range in which the limit of 5% of the maximum value is not exceeded, can alternatively already begin at 296 nm, that is to say can comprise the range from 296 nm to 329 nm. The limit of 5% can, optionally, also be chosen to be lower, for example 3.5% of the maximum value, 2% of the maximum value or even 1% of the maximum value. Said limitations of the spectral irradiance E_{λ}(λ) likewise contribute to keeping the ratio between E_{NMSC} and Eₑᵣ low, the range of a mercury peak being included.

In one embodiment, the spectral irradiance E_{λ}(λ) has a maximum value in the wavelength range from 330 nm to 364 nm, and has values for wavelengths greater than 364 nm, which do not exceed 5% of said maximum value. The limit of 5% can, optionally, also be chosen to be lower, for example 3.5% of the maximum value, 2% of the maximum value or even 1% of the maximum value. A further wavelength range, in which the ratio between E_{NMSC} and Eₑᵣ is unfavorable, is eliminated by said limitations of the spectral irradiance E_{λ}(λ).

In principle, the radiation source can have any type and number of radiation bodies, including combinations of different types. In one embodiment, the radiation source has a low-pressure UV lamp. The spectrum of the radiation which is produced can be influenced by the composition of the luminescent substance contained. The luminescent substance composition can contain, e.g., phosphor in different chemical compounds.

Likewise, a glass of the low-pressure UV lamp, which encloses the luminescent substance composition, can be utilized as a filter, by way of example by applying a suitable coating. It is therefore easily possible to attain the desired ratio between E_{NMSC} and Eₑᵣ by having recourse to the proven technology of fluorescent lamps.

In one embodiment, the radiation source has a high-pressure UV burner. In the case of high-pressure UV burners, the emitted spectrum likewise results from the properties of the lighting medium which can be influenced by doping, as a general rule in combination with a filter disk. Similarly to a low-pressure UV lamp, it is therefore easily possible to choose the spectral irradiance E_{λ}(λ) such that the desired ratio between E_{NMSC} and Eₑᵣ is achieved. Likewise, a glass of the low-pressure UV lamp, which encloses the luminescent substance composition, can be utilized as a filter, since the composition of the glass permits a differentiated emission of the radiation such that in combination with the luminescent substance composition the desired ratio between E_{NMSC} and Eₑᵣ is attained. To this end, proven technology can be utilized.

In one embodiment, the radiation source has a UV LED. LED stands for light-emitting diode. In the case of UV LEDs, the UV radiation is produced by recombining electrons and holes as a consequence of transitions of electrons from the conduction band into the valence band. The band gap between the conduction band and valence band establishes the wavelength. The width of the conduction and valence bands and the distance thereof can be deliberately influenced by selecting and doping the semiconductor materials used. Of course, the radiation source can have a plurality of UV LEDs, in particular a plurality of UV LEDs having different wavelengths. Consequently, the spectral irradiance E_{λ}(λ) can also be chosen such that the desired ratio between E_{NMSC} and Eₑᵣ is achieved by using UV LEDs.

In one embodiment, the radiation source has a filter disk. As a result, specific wavelength ranges can be deliberately filtered out of the generated radiation. The material of the filter disk can be matched to the wavelength range to be filtered out, and multiple filter disks can also be combined. The version with a filter disk makes particular sense in combination with a high-pressure UV burner, but is not limited thereto.

The object indicated above is likewise achieved by the skin irradiation device for tanning skin having the advantages of Claim 9. It comprises at least one radiation source according to any one of the Claims 1 to 8.

In one embodiment, the biologically effective irradiance for the direct pigmentation Eₚᵢ is at least 180 W/m², preferably at least 200 W/m². Said indication refers to a distance between the skin and the radiation source which is complied with when the skin irradiation device is in operation. A good tanning effect is attained by the indicated irradiance.

In one embodiment, the erythema-effective, biologically effective irradiance Eₑᵣ does not exceed 1.0 W/m², preferably does not exceed 0.3 W/m². Said indication also refers to a distance between the skin and the radiation source which is complied with when the skin irradiation device is in operation. Excessive irradiation is avoided.

In one embodiment, the skin irradiation device is a solarium, in particular a sunbed or a sun shower.

The invention is explained in greater detail below, with reference to exemplary embodiments depicted in figures, wherein:

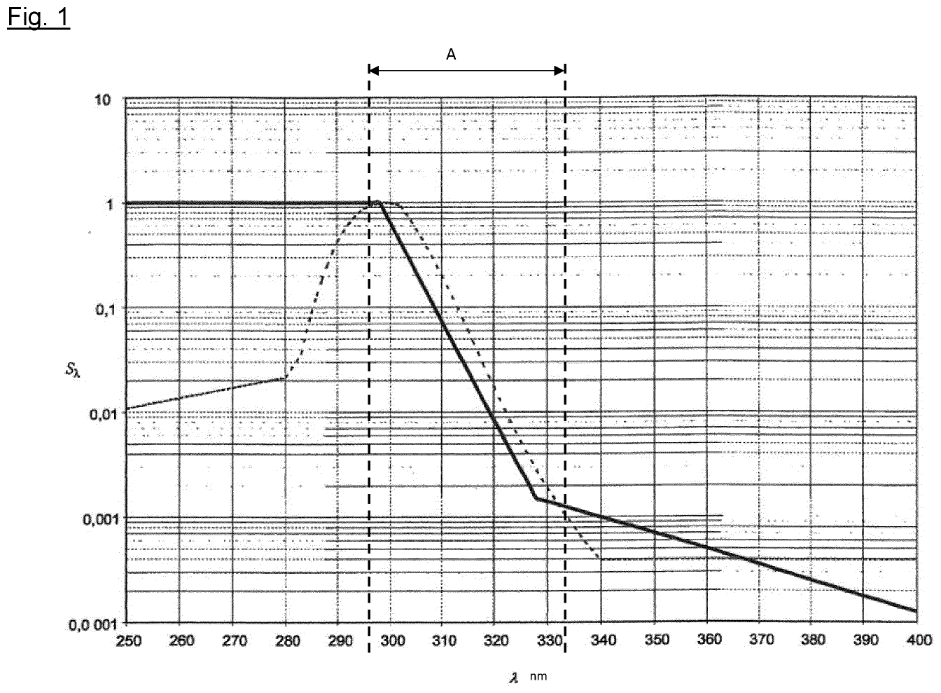

FIG. 1 shows a diagram with the relative spectral sensitivities for the UV erythema and for NMSC,

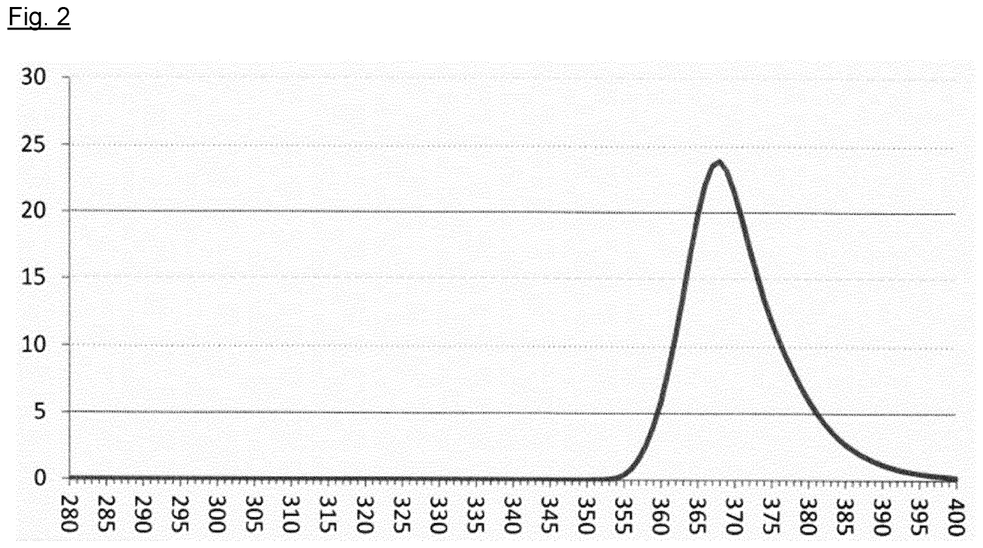

FIG. 2 shows a diagram of the spectral irradiance E_{λ}(λ) of a LED shoulder tanner according to the prior art,

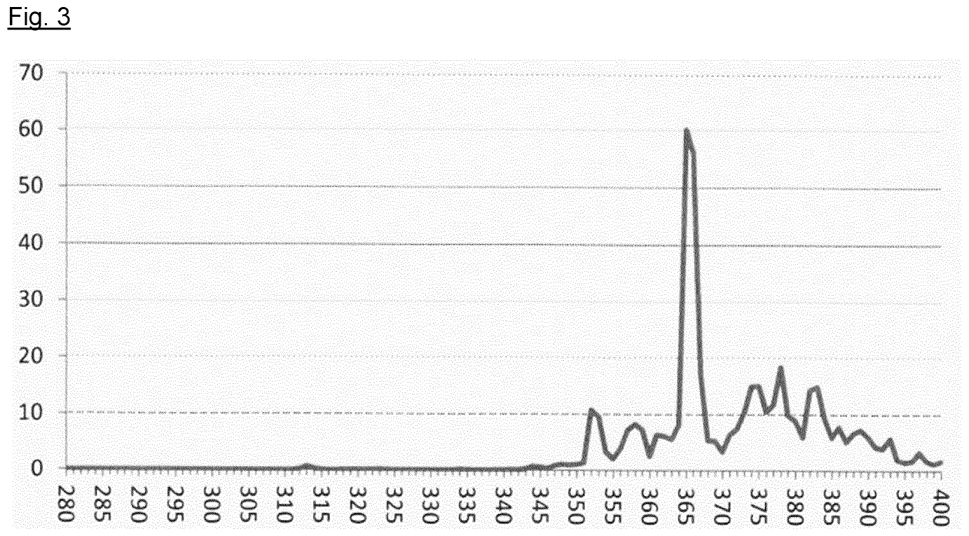

FIG. 3 shows a diagram of the spectral irradiance E_{λ}(λ) of a high-pressure radiator having a filter disk according to the prior art,

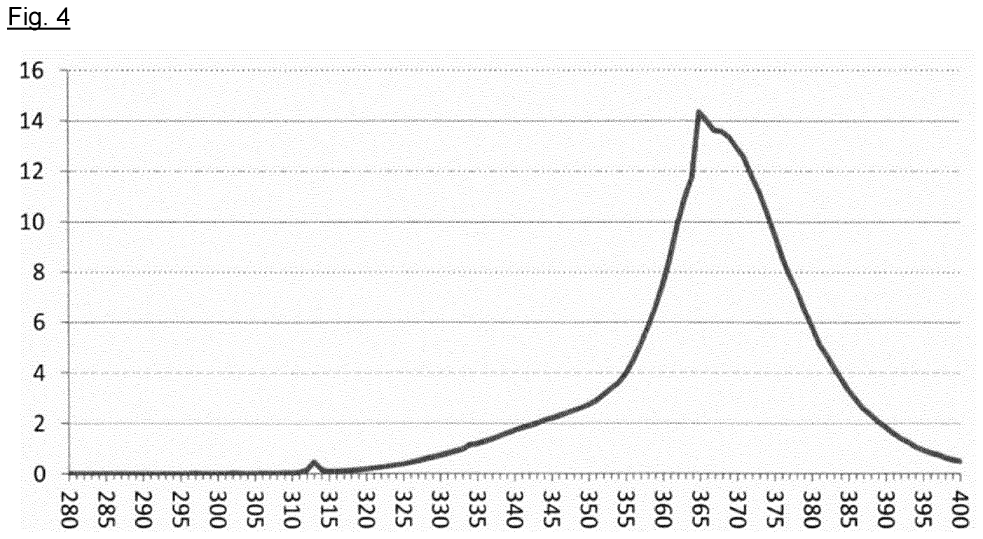

FIG. 4 shows a diagram of the spectral irradiance E_{λ}(λ) of low-pressure UV lamps according to the prior art,

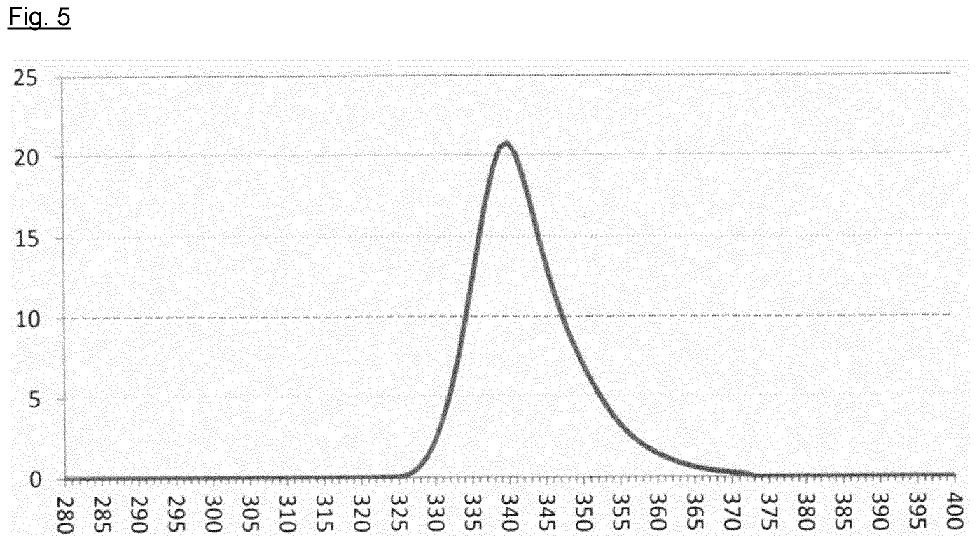

FIG. 5 shows a diagram of the spectral irradiance E_{λ}(λ) of a UV LED according to the invention.

The relative spectral sensitivities S_{λ} for the UV erythema and for NMSC are plotted over the wavelength k in nanometers in FIG. 1. The designation A_{boil}(λ) was used above for the same variables. The relative spectral sensitivity S_{λ} is normalized and has no units and is plotted logarithmically in FIG. 1. The dashed curve shows the relative spectral sensitivity for NMSC, the solid line shows that for UV erythema. It is obvious that, in particular, in the wavelength range between approx. 296 nm and 333 nm, which is identified with λ the action functions for the erythema as well as for NMSC lie very close to one another, NMSC even exceeding erythema. The two action functions are indicated in tabular form in the DIN 5031-10:2018-03 standard.

FIGS. 2 to 5 each show a diagram of a spectral irradiance E_{λ}(λ) of a UV radiation source, indicated or, respectively measured at the distance provided during the tanning of the skin. The power in W/m² striking in a wavelength interval Δλ of 1 nm is plotted over the wavelength k in nm. On said basis, the erythema-effective irradiance Eₑᵣ and the NMSC-effective irradiance E_{NMSC} were calculated for each of the UV radiation sources, by forming the following sum:

$${E}_{eff} = {\sum }_{250 nm}^{400 nm} {S}_{λ} {E}_{λ} {Δ}_{λ}$$

Accordingly, as specified in the standard mentioned, the respective spectral sensitivity was multiplied by the spectral irradiance for each wavelength interval and the products were added up.

The spectral irradiance E_{λ}(λ) from FIG. 2 was measured with a double monochromator on a Blue Vision-branded LED shoulder tanner. The determined NMSC-effective irradiance E_{NMSC} is 0.139 W/m², the erythema-effective irradiance Eₑᵣ is 0.126 W/m². The ratio of E_{NMSC} to Eₑᵣ is 1.10.

The spectral irradiance E_{λ}(λ) from FIG. 3 was measured with a double monochromator on a high-pressure UV radiator having a filter disk (Prestige 1400, Filter disk Type 3). The determined NMSC-effective irradiance E_{NMSC} is 0.464 W/m², the erythema-effective irradiance Eₑᵣ is 0.299 W/m². The ratio of E_{NMSC} to Eₑᵣ is 1.55.

The spectral irradiance E_{λ}(λ) from FIG. 4 was measured with a double monochromator on low-pressure UV lamps (Ergo 1400 New Technology x-Tend plus with upper part). The determined NMSC-effective irradiance E_{NMSC} is 0.408 W/m², the erythema-effective irradiance Eₑᵣ is 0.291 W/m². The ratio of E_{NMSC} to Eₑᵣ is 1.40.

FIG. 5 shows the spectral irradiance E_{λ}(λ) of a radiation source according to the invention, which has a UV LED. The radiation emitted extends over a wavelength range from approximately 325 nm to approximately 372 nm and has a maximum value of 20.8 W/m² at 340 nm. The determined NMSC-effective irradiance E_{NMSC} is 0.170 W/m², the erythema-effective irradiance Eₑᵣ is 0.292 W/m². The ratio of E_{NMSC} to Eₑᵣ is 0.59.

Further exemplary embodiments of the invention are indicated below in the form of numbered paragraphs:
1. A radiation source for emitting UV radiation for a skin irradiation device for tanning skin, characterized by a ratio of the NMSC-effective irradiance (E_{NMSC}) to the erythema-effective irradiance (Eₑᵣ) of less than or equal to 0.9 (E_{NMSC}/Eₑᵣ≤0.9).
2. The radiation source according to paragraph 1. characterized in that the ratio is substantially less than or equal to 0.9 over the entire wavelength range relevant to the skin tanning.
3. The radiation source according to paragraph 1 or 2, characterized in that the ratio is substantially less than or equal to 0.9 over a wavelength range from approx. 280 nm to approx. 340 nm.
4. The radiation source according to any one of the preceding paragraphs, characterized in that the radiation source is designed to emit substantially no/hardly any radiation in a wavelength range from approx. 296 nm to approx. 333 nm.
5. The radiation source according to any one of the preceding paragraphs, characterized in that the radiation source is designed to emit no/hardly any radiation as of a wavelength of 340 nm upwards.
6. The radiation source according to any one of the preceding paragraphs, characterized by a ratio of vitamin D3-effective irradiance (E_{D3}) to erythema-effective irradiance (Eₑᵣ) of at least 0.14.
7. The radiation source according to any one of the preceding paragraphs, characterized in that the vitamin-D3-effective irradiance (E_{D3}) is at least 0.03 W/m².
8. The radiation source according to any one of the preceding paragraphs, characterized in that the radiation source comprises a low-pressure UV lamp.
9. The radiation source according to paragraph 8, characterized in that the low-pressure UV lamp is designed to only emit radiation in a wavelength range relating to the mercury peak in a wavelength range from approx. 296 nm to approx. 333 nm.
10. The radiation source according to any one of the preceding paragraphs, characterized in that the radiation source comprises a high-pressure UV burner, in particular having a filter disk.
11. The radiation source according to any one of the preceding paragraphs, characterized in that the radiation source has a UV LED.
12. The radiation source according to any one of the preceding paragraphs, characterized by an irradiance for the direct pigmentation (Eₚᵢ) of at least 180 watts per square meter, preferably of at least 200 watts per square meter.
13. A skin irradiation device for tanning skin, comprising at least one radiation source according to any one of the preceding paragraphs.
14. The skin irradiation device according to paragraph 13, characterized in that the skin irradiation device is a solarium, in particular a sunbed or a sun shower.

1. A solarium for tanning skin with a radiation source, designed to emit UV light at a spectral irradiance Eλ(λ), the biologically effective irradiance for the direct pigmentation Epi being at least 180 W/m2, characterized in that the spectral irradiance Eλ(λ) is chosen such that the ratio of the NMSC-effective, biological irradiance ENMSC to the erythema-effective, biological irradiance Eer is 0.9 or less.

2. The solarium according to claim 1, characterized in that the spectral irradiance Eλ(λ) has a maximum value in the wavelength range from 330 nm to 390 nm, and has values in the wavelength range from 299 nm to 311 nm and in the wavelength range from 315 nm to 329 nm, which do not exceed 5% of said maximum value.

3. The solarium according to claim 1, characterized in that the spectral irradiance Eλ(λ) has a maximum value in the wavelength range from 330 nm to 390 nm, and has values in the wavelength range from 299 nm to 329 nm, which do not exceed 5% of said maximum value.

4. The solarium according to claim 1, characterized in that the spectral irradiance Eλ(λ) has a maximum value in the wavelength range from 330 nm to 364 nm, and has values for wavelengths greater than 364 nm, which do not exceed 5% of said maximum value.

5. The solarium according to claim 1, characterized in that the radiation source has a low-pressure UV lamp.

6. The solarium according to claim 1, characterized in that the radiation source has a high-pressure UV burner.

7. The solarium according to claim 1, characterized in that the radiation source has a UV LED.

8. The solarium according to claim 1, characterized in that the radiation source has a filter disk.

9. The solarium according to claim 1, characterized in that the biologically effective irradiance for the direct pigmentation Epi is at least 200 W/m2.

10. The solarium according to claim 1, characterized in that the erythema-effective, biologically effective irradiance Eer does not exceed 1.0 W/m2, preferably does not exceed 0.3 W/m2.

11. The solarium according to claim 19, characterized in that the solarium is a sunbed or a sun shower.
